(19) United States
(12) Patent Application Publication
(10) Pub. No.: US 2013/0053410 A1
(43) Pub. Date: Feb. 28, 2013

(54) SUBSTITUTED HETEROARYL 2',3',7',7A'-TETRAHYDROSPIRO[PYRROLE-3,6'-PYRROLO[1,2-C]IMIDAZOLE]-1',2(1H,5'H)-DIONE

(71) Applicants: David Joseph Bartkovitz, Nutley, NJ (US); Xin-Jie Chu, Livingston, NJ (US); Qingjie Ding, Bridgewater, NJ (US); Nan Jiang, Pine Brook, NJ (US); Jin-Jun Liu, Warren Township, NJ (US); Zhuming Zhang, Hillsborough, NJ (US)

(72) Inventors: David Joseph Bartkovitz, Nutley, NJ (US); Xin-Jie Chu, Livingston, NJ (US); Qingjie Ding, Bridgewater, NJ (US); Nan Jiang, Pine Brook, NJ (US); Jin-Jun Liu, Warren Township, NJ (US); Zhuming Zhang, Hillsborough, NJ (US)

(21) Appl. No.: 13/400,296

(22) Filed: Feb. 20, 2012

Related U.S. Application Data
(60) Provisional application No. 61/448,687, filed on Mar. 3, 2011.

(51) Int. Cl. C07D 471/22; A61P 35/00; A61K 31/437

(52) U.S. Cl. 514/278; 546/15

(57) ABSTRACT

There are provided compounds of the formula

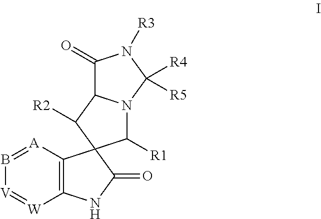

wherein A, B, V, W, R¹, R², R³, R⁴ and R⁵ are described herein together with the enantiomers and pharmaceutically acceptable salts and esters thereof. The compounds are useful as anticancer agents.

### PRIORITY TO RELATED APPLICATION(S)

This application claims the benefit of U.S. Provisional Application No. 61/448,687, filed Mar. 3, 2011, which is hereby incorporated by reference in its entirety.

### FIELD OF THE INVENTION

The present invention relates to substituted heteroaryl 2′,3′,7′,7a′-tetrahydrospiro[pyrrole-3,6′-pyrrolo[1,2-c]imidazole]-1′,2(1H,5′H)-dione derivatives which act as inhibitors of MDM2-p53 interactions and are useful in the amelioration or treatment of cancer.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

### SUMMARY OF THE INVENTION

The present invention relates to substituted heteroaryl 2′,3′,7′,7a′-tetrahydrospiro[pyrrole-3,6′-pyrrolo[1,2-c]imidazole]-1′,2(1H,5′H)-dione derivatives of formula I which act as antagonists of MDM2 interactions and hence are useful as potent and selective anticancer agents. The present compounds are of the general formula

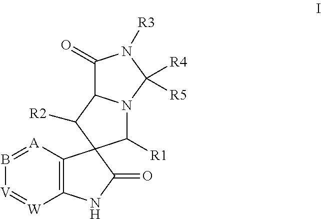

wherein A, B, V, W, R¹, R², R³, R⁴ and R⁵ are described herein and enantiomers and pharmaceutically acceptable salts and esters thereof.

### DETAILED DESCRIPTION OF THE INVENTION

There are provided compounds of the formula

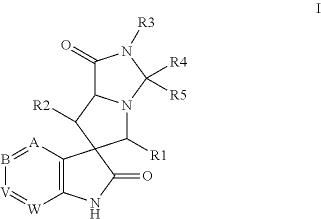

wherein

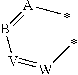

is selected from the group consisting of

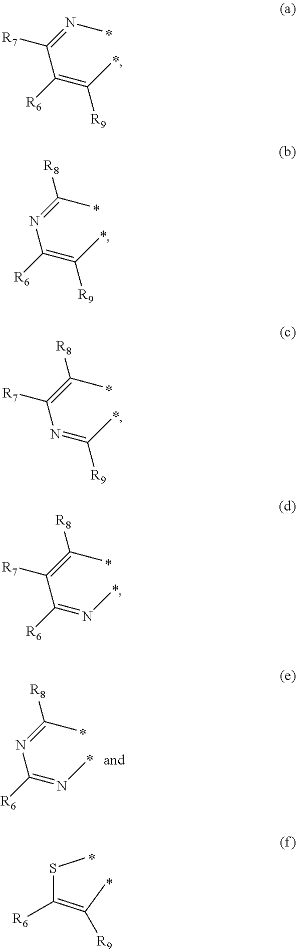

R₆ is selected from the group consisting of H, F, Cl, Br, I, CN, NO₂, ethynyl, cyclopropyl, lower alkyl, vinyl and alkoxy,
R₇ is selected from the group consisting of H, F, Cl, methyl;
R₈ is selected from the group consisting of H, F, Cl, methyl;
R₉ is selected from the group consisting of H, F, Cl, methyl;
wherein in the case of (f) A is a bond;
R₁ is independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ is selected from the group consisting of aryl, substituted aryl, heteroaryl and substituted heteroaryl;
R₃ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′ wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
and enantiomers thereof or a pharmaceutically acceptable salt or ester thereof.

Another embodiment of the invention relates to compounds of formula I having a stereochemical structure shown as formula II

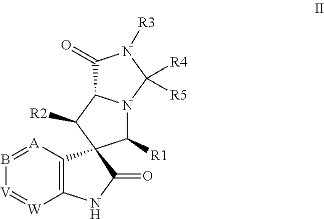

wherein

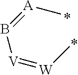

is selected from the group consisting of

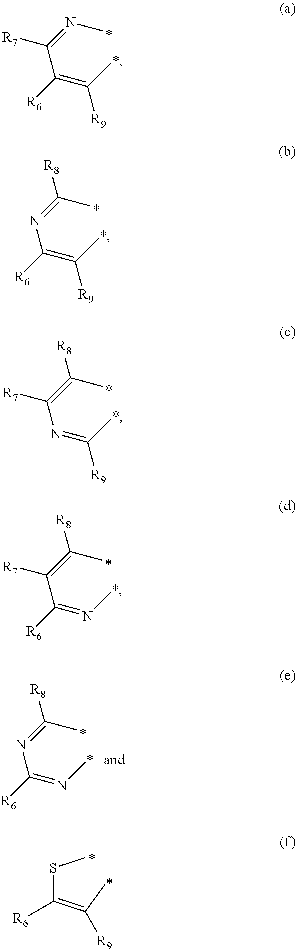

R₆ is selected from the group consisting of H, F, Cl, Br, I, CN, NO₂, ethynyl, cyclopropyl, lower alkyl, vinyl and alkoxy;
R₇ is selected from the group consisting of H, F, Cl, methyl;
R₈ is selected from the group consisting of H, F, Cl, methyl;
R₉ is selected from the group consisting of H, F, Cl, methyl;
wherein in the case of (f) A is a bond;
R₁ is independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ is selected from the group consisting of aryl, substituted aryl, heteroaryl and substituted heteroaryl;
R₃ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ— (CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′ wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
and enantiomers thereof or a pharmaceutically acceptable salt or ester thereof.

Preferred are compounds of Formula I including compounds of Formula II or a pharmaceutically acceptable salt thereof wherein R₆ is F, Cl or Br.

Preferred are compounds of Formula I including compounds of Formula II or a pharmaceutically acceptable salt thereof wherein R₇, R₈, R₉ are all hydrogen.

Preferred are compounds of Formula I including compounds of Formula II or a pharmaceutically acceptable salt thereof wherein R₂ is selected from the group consisting of aryl, aryl substitued with Cl or F or Br and heteroaryl optionally substituted with H, F, Cl or Br.

Preferred are compounds of Formula I including compounds of Formula II or a pharmaceutically acceptable salt thereof wherein R₁ is a substituted lower alkyl of the formula

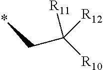

where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2 and
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle.

Preferred are compounds of Formula I including compounds of Formula II or a pharmaceutically acceptable salt thereof wherein one of R₃ is (CH₂)ₙ—R′, n is 0 or 1 and R′ is aryl, substituted aryl, hetereoaryl, substituted heteroaryl, hetereocycle or substituted heterocycle.

Preferred are compounds of Formula I including compounds of Formula II or a pharmaceutically acceptable salt thereof wherein

R₆ is selected from F, Cl or Br;
R₇, R₈, R₉ are hydrogen;
R₂ is selected from the group consisting of aryl, aryl substitued with Cl or F or Br, and heteroaryl optionally substituted with H, F or Cl or Br;
R₁ is a substituted lower alkyl of the formula

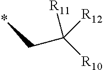

where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2;
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
R₃ is (CH₂)ₙ—R′;
n is 0 or 1 and
R′ is selected from aryl, substituted aryl, hetereoaryl, substituted heteroaryl, hetereocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
and enantiomers thereof or a pharmaceutically acceptable salt, or ester thereof.

Further preferred are compounds of Formula II

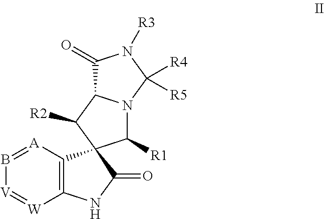

wherein

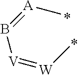

is selected from the group consisting of

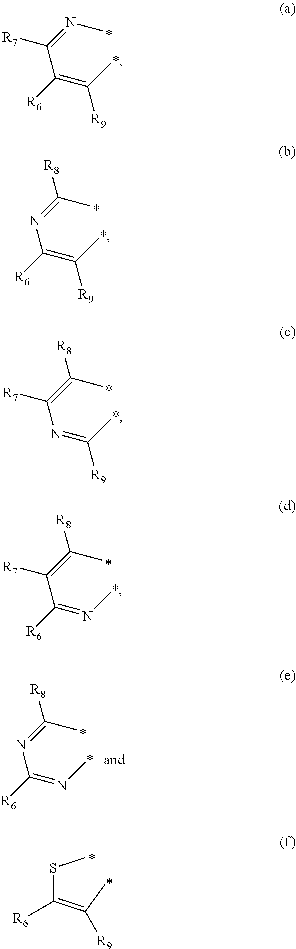

R₆ is selected from F, Cl or Br;
R₇, R₈, R₉ are hydrogen;
R₂ is selected from the group consisting of

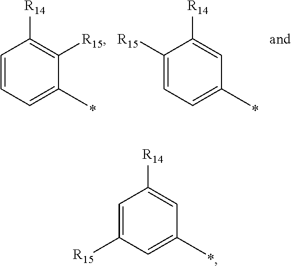

wherein

### R₁₄ is F, Cl or Br;

### R₁₅ is H or F;

R₁ is a substituted lower alkyl of the formula

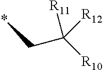

where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2;
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
R₃ is (CH₂)ₙ—R′;
n is 0 or 1;
R′ is selected from aryl, substituted aryl, hetereoaryl, substituted heteroaryl, hetereocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
or a pharmaceutically acceptable salt thereof.

Especially preferred are compounds of the formulas
chiral 4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′S,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid,
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-2-methoxybenzamide,
rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid,
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-ethyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide;
chiral 4-((3S,3′R,5S,7S,7aR)-6′-chloro-7-(3-chloro-2-fluorophenyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide;
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid and
rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid.

In the specification where indicated the various groups may be substituted by 1-5 or, preferably, 1-3 substituents independently selected from the group consisting of lower alkyl, lower-alkenyl, lower-alkynyl, dioxo-lower-alkylene (forming e.g. a benzodioxyl group), halogen, hydroxy, CN, CF₃, NH₂, N(H, lower-alkyl), N(lower-alkyl)₂, aminocarbonyl, carboxy, NO₂, lower-alkoxy, thio-lower-alkoxy, lower-alkylsulfonyl, aminosulfonyl, lower-alkylcarbonyl, lower-alkylcarbonyloxy, lower-alkoxycarbonyl, lower-alkyl-carbonyl-NH, fluoro-lower-alkyl, fluoro-lower-alkoxy, lower-alkoxy-carbonyl-lower-alkoxy, carboxy-lower-alkoxy, carbamoyl-lower-alkoxy, hydroxy-lower-alkoxy, NH₂-lower-alkoxy, N(H, lower-alkyl)-lower-alkoxy, N(lower-alkyl)-2-lower-alkoxy, lower-alkyl-1-oxiranyl-lower-alkoxy-lower-alkyl, 2-oxo-pyrrolidin-1-yl, (1,1-dioxo)-2-isothiazolidine, 3-lower-alkyl sulfinyl, a substituted or unsubstituted heterocyclic ring, a substituted or unsubstituted aryl ring, a substituted or unsubstituted heteroaryl ring, trifluoro-lower-alkylsulfonylamino-aryl, lower-alkyl sulfonylaminocarbonyl, lower-alkyl sulfonylaminocarbonyl-aryl, hydroxycarbamoyl-phenyl, benzyloxy-lower-alkoxy, mono- or di-lower alkyl substituted amino-sulfonyl and lower-alkyl which can optionally be substituted with halogen, hydroxy, NH₂, N(H, lower-alkyl) or N(lower-alkyl)₂. Preferred substituents for the cycloalkyl, cycloalkenyl, aryl, heteroaryl and heterocycle rings are halogen, lower alkoxy, lower alkyl, hydroxycarbonyl, carboxy, carboxy lower alkoxy, oxo and CN. Preferred substituents for alkyl are alkoxy and N(lower alkyl)₂.

### Terms & Definitions

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle;

The term “alkyl” refers to straight- or branched-chain saturated hydrocarbon groups having from 1 to about 20 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, and in certain embodiments from 1 to 4 carbon atoms. Examples of alkyl groups include, but are not limited to, methyl, ethyl, n-propyl, i-propyl, n-butyl, s-butyl, t-butyl, n-pentyl, and s-pentyl.

As used herein, “cycloalkyl” is intended to refer to any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “alkenyl” as used herein means an unsaturated straight-chain or branched aliphatic hydrocarbon group containing one double bond and having 2 to 6, preferably 2 to 4 carbon atoms.

Examples of such “alkenyl group” are vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “halogen” as used in the definitions means fluorine, chlorine, bromine, or iodine, preferably fluorine and chlorine.

“Aryl” means a monovalent, monocyclic or bicyclic, aromatic carbocyclic hydrocarbon radical, preferably a 6-10 member aromatic ring system. Preferred aryl groups include, but are not limited to, phenyl, naphthyl, tolyl, and xylyl.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole and tetrazolyl. In the case of aryl or heteroaryl which are bicyclic it should be understood that one ring may be aryl while the other is heteroaryl and both being substituted or unsubstituted.

“Heterocycle” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like.

“Hetero atom” means an atom selected from N, O and S.

“Alkoxy, alkoxyl or lower alkoxy” refers to any of the above lower alkyl groups attached to an oxygen atom. Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (6th Ed. 1995) at pp. 196 and 1456-1457.

The compounds of formula I and II as well as their salts that have at least one asymmetric carbon atom may be present as racemic mixtures or different stereoisomers. The various isomers can be isolated by known separation methods, e.g., chromatography.

Compounds disclosed herein and covered by formula I and II above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

The compounds of the present invention are useful in the treatment or control of cell proliferative disorders, in particular oncological disorders. These compounds and formulations containing said compounds may be particularly useful in the treatment or control of solid tumors, such as, for example, breast, colon, lung and prostate tumors.

A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

“Effective amount” means an amount that is effective to prevent, alleviate or ameliorate symptoms of disease or prolong the survival of the subject being treated.

“IC50” refers to the concentration of a particular compound required to inhibit 50% of a specific measured activity. IC₅₀ can be measured, inter alia, as is described subsequently.

### Synthetic Methods

The present invention provides novel methods for the synthesis of the substituted heteroaryl 2′,3′,7′,7a′-tetrahydrospiro[pyrrole-3,6′-pyrrolo[1,2-c]imidazole]-1′,2(1H,5′H)-dione derivatives of formula I or II. Compounds of this invention can be synthesized according to the following general schemes. The detailed processes for synthesizing these compounds are provided in the examples.

An intermediate III can be made from a base-catalyzed condensation reaction of appropriately selected substituted 4- or 5- or 6- or 7-aza-2-oxindole I and appropriate substituted aldehyde II in methanol (Scheme 1). The choice of bases includes but is not limited to pyrrolidine or piperidine. The reaction generates III as a mixture of Z- and E-isomers with E-isomer as the major product.

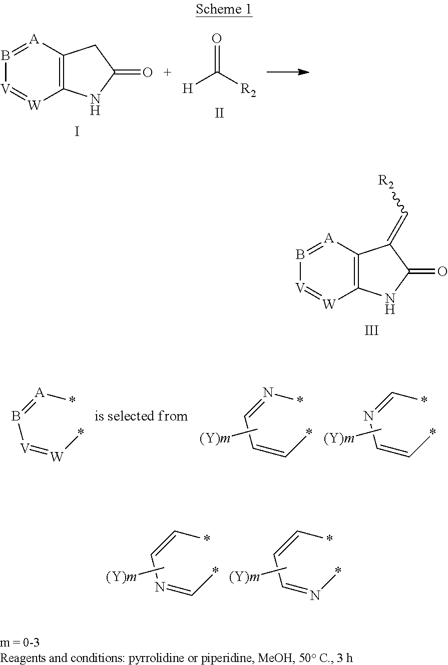

Similarly, intermediates V or VII can be made from a acid-catalyzed condensation reaction of appropriately selected substituted 5,7-dihydro-pyrrolo[2,3-d]pyrimidin-6-one IV or 4,6-dihydro-thieno[3,2-b]pyrrol-5-one VI and aldehyde II in hydrochloric and acetic aicd (M. Cheung et al, Tetrahedron Lett. 2001, 42, 999) (Scheme 2).

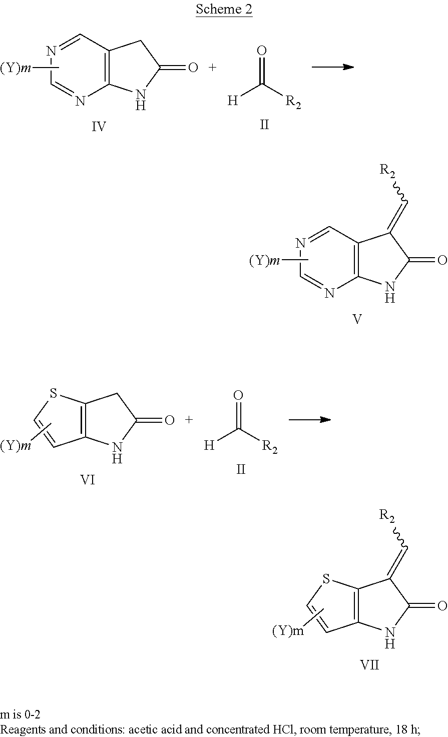

Preparation of starting material VIa is described in Scheme 3 to exemplify the synthesis of intermediate VI in Scheme 2. 2,5-Dichlorothiophene can be treated with sodium nitrate in concentrated sulfuric acid to give 2,5-dichlorothiphene-3-nitrothiophene. Nucleophilic substitution of 5-chloro group with tert-butyl ethyl malonate mono-sodium salt and treatment with trifluoroacetic acid lead to methyl 2-(5-chloro-3-nitrothiophen-2-yl)acetate (WO2008132139). Reduction of nitro group with zinc and ammonium chloride and cyclizing reaction to form amide promoted by trimethylaluminum afford intermediate VIa (S. Hu, et al, J. Heterocyclic. Chem. 2005, 42, 661).

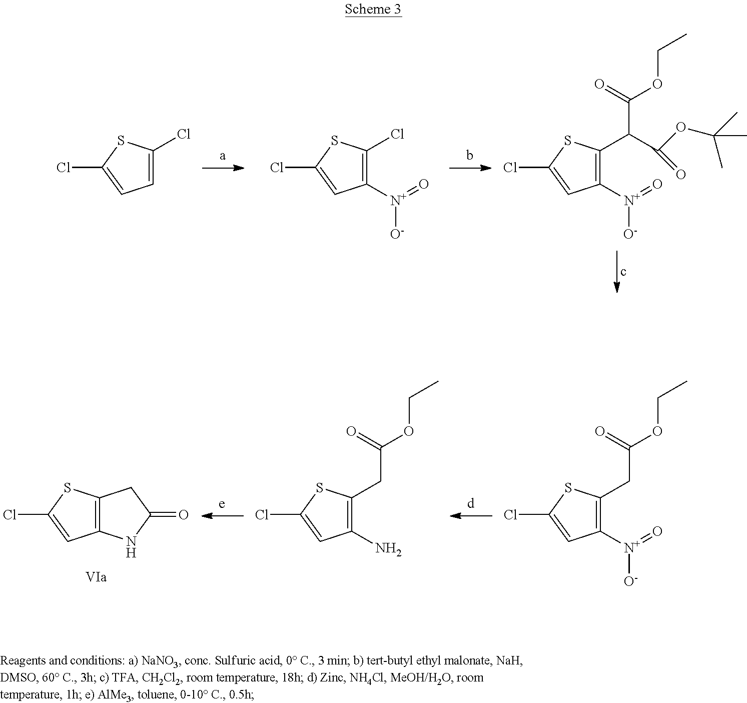 [SMILES: Clc1ccc(Cl)s1 ; O=[N+]([O-])c1cc(Cl)sc1Cl ; CCOC(=O)C(C(=O)OC(C)(C)C)c1sc(Cl)cc1[N+](=O)[O-] ; O=C1Cc2sc(Cl)cc2N1 ; CCOC(=O)Cc1sc(Cl)cc1N ; CCOC(=O)Cc1sc(Cl)cc1[N+](=O)[O-]]

Racemic synthesis of compounds XI and XII can be achieved as outlined in Scheme 4. Amine R₃NH₂ can be reacted with N-protected glycine like N-Boc glycine by using a coupling reagent like EDCI or HATU to give intermediate VIII. Intermediate VIII can be treated with trifluoroacetic acid or HCl at room temperature to remove protective Boc group and give intermediate IX. Appropriately selected aldehyde RiCHO can react with IX to give the imine X. The cylcoaddition reaction between intermediates X and intermediate III or V or VII mediated by LiOH or LiCl/DABCO gives a racemic and diastereomeric mixture of compounds XI together with other isomers. Compounds XI can be purified by flash chromatography followed by chiral separation by chiral Super Fluid Chromatography (SFC) or chiral HPLC to give oprically pure or enriched chiral compounds XII.

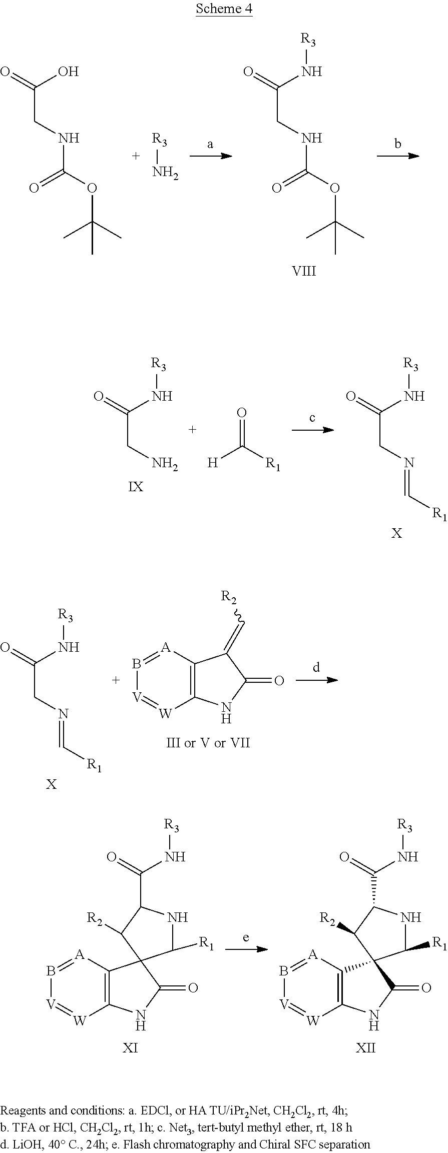

Similarly, compounds XI and XII can be prepared as outlined in Scheme 5. Intermediate III or V or VII can be protected with Boc group to give intermediate XIII. The cylcoaddition reaction between intermediates X and XIII mediated by LiOH or LiCl/DABCO follow by reaction to remove Boc group by trifluoroacetic aicd give compounds XI. Compounds XI can be subsequently separated into oprically pure or enriched chiral compounds XII.

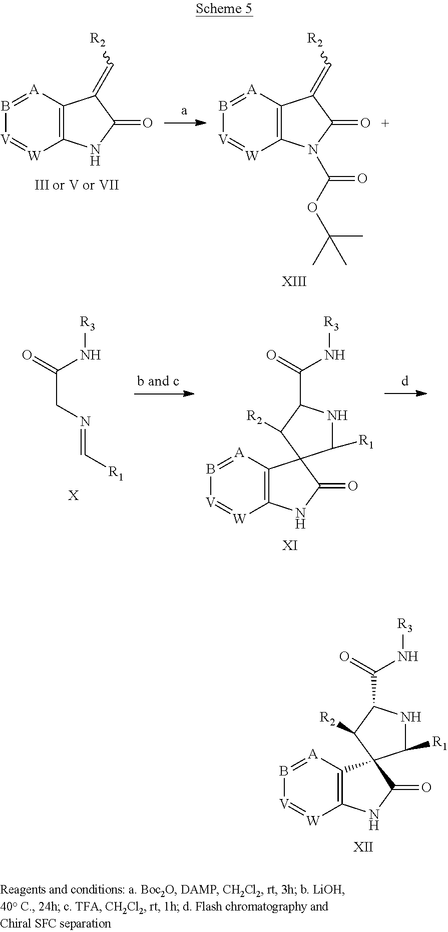

Alternative synthesis of compounds XIIa can be achieved. As illustrated in Scheme 6, selected aldehyde R₁CHO can be reacted with glycine tert-butyl ester to generate imine XIV. The racemic mixture of intermediate XV and XV′ can be made from intermediates XIV and VII by LiOH mediated cyclization reaction. The mixture of XVI and XVI′ can be subsequently converted to a racemic mixture of acid XVII and XVII′ by using trifluoroacetic acid. Amide formation with various amine NHR₃R₄ by using diphenylphosphinic chloride as the coupling reagent can lead to the racemic mixture of compounds XIIa and XIIa′. Finally chiral separation by chiral Super Fluid Chromatography (SFC) or chiral HPLC gives optically pure or enriched chiral compounds XIIa.

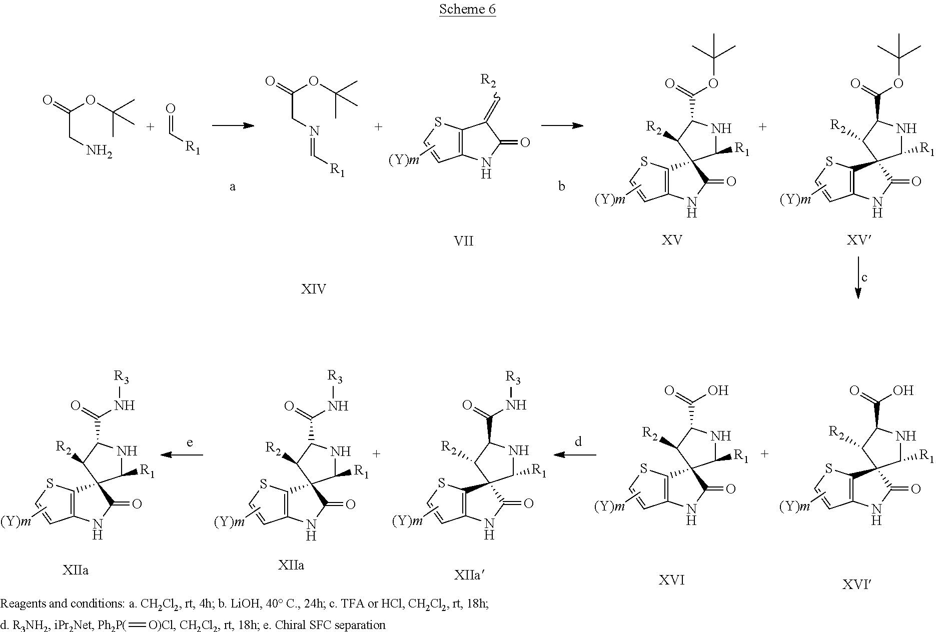

Synthesis of compounds XVIII and XIX in formula I or II can be achieved by the reaction of intermediates XI or XII with a variety of aldehydes or ketones XVII in acidic condition (preferably acetic acid) followed by aqueous basic workup (Scheme 7).

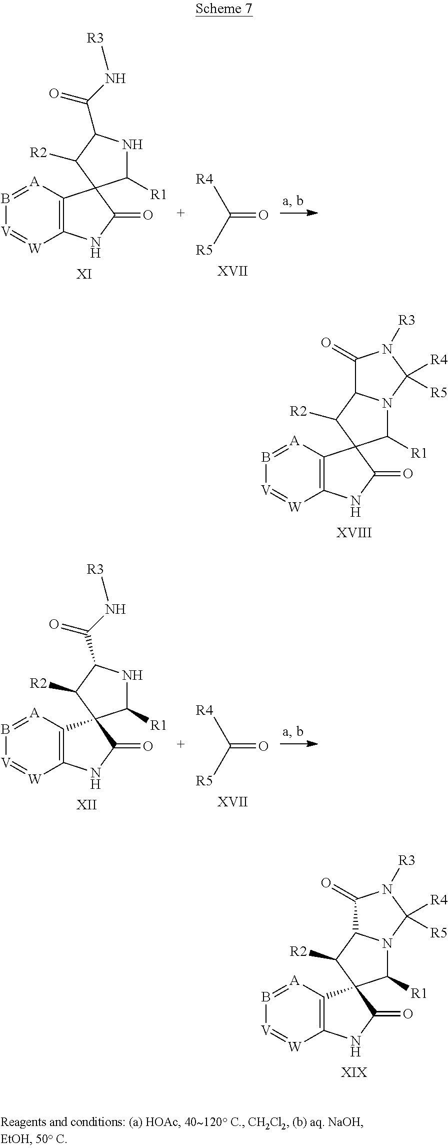

### EXAMPLES

The compounds of the present invention may be synthesized according to novel techniques. The following examples and references are provided to aid the understanding of the present invention, the true scope of which is set forth in the appended claims.

### Example 1

### Preparation of chiral 4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

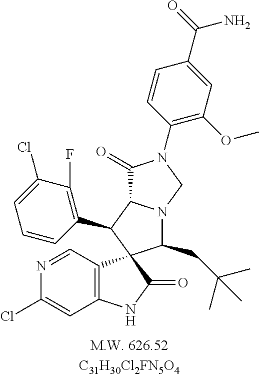 [SMILES: COc1cc(C(N)=O)ccc1N1CN2[C@@H](CC(C)(C)C)[C@@]3(C(=O)Nc4cc(Cl)ncc43)[C@@H](c3cccc(Cl)c3F)[C@@H]2C1=O]

Step a: To a solution of chiral (2S,3R,4S,5R)—N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide (24 mg, 0.039 mmol) and acetic acid (0.4 mL) in dichloromethane (0.4 mL), was added formaldehyde (contains 10-15% methanol as inhibitor) (27 mg, 0.02 mL, 0.336 mmol). The reaction mixture was heated under argon at 40° C. overnight. It was then poured into 2 N NaOH (4 mL) and water (5 mL) and extracted with EtOAc (10 mL). The organic layer was washed with water (8 mL) and concentrated to give a crude film, chiral 4-((3′R,5S,7S,7aR)-6′-chloro-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (24 mg, 95 yield).

Step b: The above crude chiral 443′R,5S,7S,7aR)-6′-chloro-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (24 mg, 0.037 mmol) was mixed with EtOH (3 mL) and treated with a solution of 2N NaOH (0.53 mL, 1.06 mmol). The reaction mixture was heated to 50° C. and stirred for 1 hr when LCMS showed the reaction was complete. The reaction mixture was poured into EtOAc (20 mL), washed with water (10 mL) and concentrated. The crude product was purified by flash chromatography (1% to 15% ethanol in dichloromethane) to give chiral 4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide as a white solid (10.6 mg, 44% yield). MS (ES⁺) m/z [(M+H)⁺]: 626

### Example 2

### Preparation of rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid

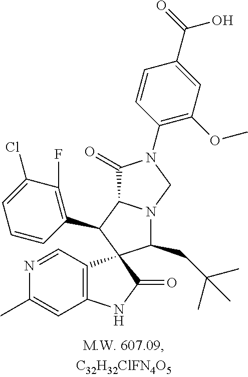

Step a: To a solution of methyl rac-442S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate (46 mg, 0.0762 mmol) and acetic acid (0.55 mL) in dichloromethane (0.6 mL), was added formaldehyde (contains 10-15% methanol as inhibitor) (33 mg, 0.03 mL, 0.403 mmol). The reaction mixture was heated under argon at 40° C. overnight. It was then poured into 2 N NaOH (4.5 mL) and water (5 mL) and extracted with EtOAc (20 mL). The organic layer was washed with water (10 mL) and concentrated to give crude methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate (61 mg) which was used without further purification.

Step b: The above crude methyl rac-4-((3′R,5S,7S,7aR)-7-(3-chloro-2-fluorophenyl)-1′-(hydroxymethyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoate (61 mg, 0.0762 mmol) was mixed with EtOH (6 mL) and treated with a solution of 2N NaOH (1.0 mL, 2.00 mmol). The reaction mixture was heated to 50° C. and stirred for 1 hr when LCMS showed the reaction was complete. The reaction mixture was neutralized with 1N HCl (2 mL to pH 6) and diluted with EtOAc (25 mL), washed with H2O (10 mL), concentrated and lyophilized from MeCN and water to give rac-4-((3′R,5S,7S,7aR)-7-(3-chloro-2-fluorophenyl)-6′-methyl-5-neopentyl-1,2′-dioxo-1′,2′,7,7a-tetrahydro spiro[pyrrolo[1,2-c]imidazole-6,3′-pyrrolo[3,2-c]pyridine]-2(1H,3H,5H)-yl)-3-methoxybenzoic acid (36 mg, 79%). MS (ES⁺) m/z [(M+H)⁺]: 607

### Example 3

### Preparation of rac-4-((3′R,5S,7S,7aR)-7-(3-chloro-2-fluorophenyl)-6′-methyl-5-neopentyl-1,2′-dioxo-1′,2′,7,7a-tetrahydro spiro[pyrrolo[1,2-c]imidazole-6,3′-pyrrolo[3,2-c]pyridine]-2(1H,3H,5H)-yl)-3-methoxybenzami de(A) and

### rac-4-((3′S,5S,7S,7aR)-7-(3-chloro-2-fluorophenyl)-6′-methyl-5-neopentyl-1,2′-dioxo-1′,2′,7,7a-tetrahydro spiro[pyrrolo[1,2-c]imidazole-6,3′-pyrrolo[3,2-c]pyridine]-2(1H,3H,5H)-yl)-3-methoxybenzamide(B)

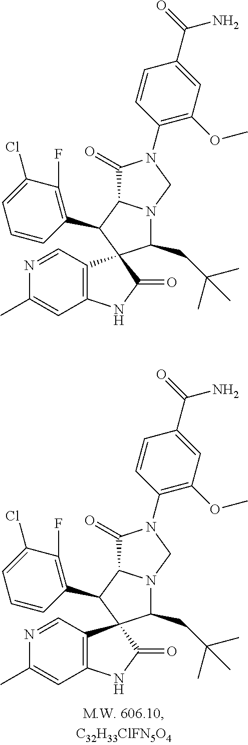

A mixture of rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid (30 mg, 0.049 mmol, Example 2) and N,N′-carbonyldiimidazole (Aldrich, 36 mg, 0.226 mmol) in THF (4 mL) was stirred at rt overnight. Ammonium hydroxide (234 mg, 0.26 mL, 6.68 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (2% to 12% EtOH in EtOAc) to give rac-443′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (A)(9 mg, 29%) as a white solid, MS (ES) m/z [(M+H)⁺]: 605, and rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (B)(17 mg, 56%) as a white solid, MS (ES) m/z [(M+H)⁺]: 606.

### Example 4

### Preparation of rac-4-((3′S,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid

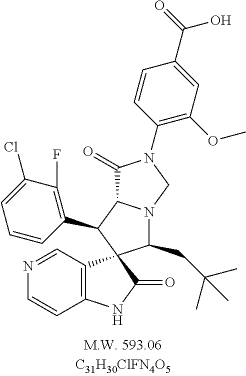 [SMILES: COc1cc(C(=O)O)ccc1N1CN2[C@@H](CC(C)(C)C)[C@@]3(C(=O)Nc4ccncc43)[C@@H](c3cccc(Cl)c3F)[C@@H]2C1=O]

Step a: To a solution of methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (50 mg, 0.084 mmol) and acetic acid (0.7 mL) in dichloromethane (0.7 mL), was added formaldehyde (contains 10-15% methanol as inhibitor) (44 mg, 0.04 mL, 0.53 mmol). The reaction mixture was heated under argon at 40° C. overnight. It was then poured into 2 N NaOH (4.5 mL) and extracted with EtOAc (2×20 mL). The organic layer was washed with water (10 mL) and concentrated to give crude methyl rac-4-((3′S,5S,7S,7aR)-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoate (52 mg) which was used without further purification.

Step b: The above crude crude methyl rac-443′S,5S,7S,7aR)-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoate (52 mg, 0.082 mmol) was dissolved in MeOH (4 mL) and treated with a solution of 2N NaOH (0.7 mL, 1.40 mmol). The reaction mixture was heated to 50° C. and stirred for 5 hr when LCMS showed the reaction was complete. The reaction mixture was neutralized with 1N HCl (1.4 mL to pH 6) and diluted with EtOAc (75 mL), washed with H2O (3×15 mL), concentrated. The crude material was absorbed on silica gel and purified by flash chromatography (silica gel, 4 g, 1% to 20% ethanol in CH₂Cl₂) to give rac-4-((3′S,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid (8.8 mg, 18% yield). MS (ES⁺) m/z [(M+H)⁺]: 593.

### Example 5

### Preparation of rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid

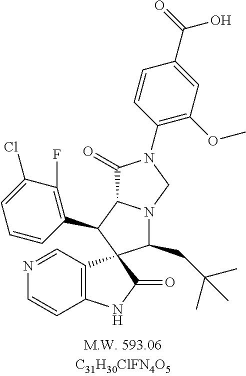 [SMILES: COc1cc(C(=O)O)ccc1N1CN2[C@@H](CC(C)(C)C)[C@@]3(C(=O)Nc4ccncc43)[C@@H](c3cccc(Cl)c3F)[C@@H]2C1=O]

Step a: To a solution of methyl rac-4-(2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (40 mg, 0.067 mmol) and acetic acid (0.5 mL) in dichloromethane (0.5 mL), was added formaldehyde (contains 10-15% methanol as inhibitor) (27 mg, 0.025 mL, 0.34 mmol). The reaction mixture was heated under argon at 40° C. overnight. It was then poured into 2 N NaOH (4.5 mL) and extracted with EtOAc (2×20 mL). The organic layer was washed with water (10 mL) and concentrated to give crude methyl rac-4-((3′R,5S,7S,7aR)-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoate (58 mg) which was used without further purification.

Step b: The above crude methyl rac-443′R,5S,7S,7aR)-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoate (58 mg, 0.067 mmol) was dissolved in EtOH (5 mL) and treated with a solution of 2N NaOH (0.9 mL, 1.80 mmol). The reaction mixture was heated to 50° C. and stirred for 2 hr when LCMS showed the reaction was complete. The reaction mixture was neutralized with 1N HCl and diluted with EtOAc (20 mL), washed with H₂O (2×10 mL), concentrated and lyophilized to give rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid (31 mg, 77%). MS (ES⁺) m/z [(M+H)⁺]: 593.

### Example 6

### Preparation of rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid

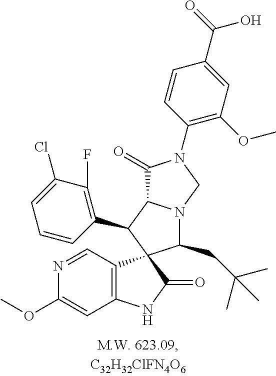

Step a: To a solution of methyl 4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate (45 mg, 0.072 mmol) and acetic acid (0.52 mL) in dichloromethane (0.5 mL), was added formaldehyde (contains 10-15% methanol as inhibitor) (32 mg, 0.030 mL, 0.40 mmol). The reaction mixture was heated under argon at 40° C. overnight. It was then poured into 2 N NaOH (4.3 mL) and extracted with EtOAc (2×20 mL). The organic layer was washed with water (10 mL) and concentrated to give crude methyl 4-((3′R,5S,7S,7aR)-7-(3-chloro-2-fluorophenyl)-1′-(hydroxymethyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoate (86 mg) which was used without further purification.

Step b: The above crude methyl 4-((3′R,5S,7S,7aR)-7-(3-chloro-2-fluorophenyl)-1′-(hydroxymethyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl-3-methoxybenzoate (86 mg, 0.072 mmol) was dissolved in EtOH (5 mL) and treated with a solution of 2N NaOH (0.95 mL, 1.90 mmol). The reaction mixture was heated to 50° C. and stirred for 2 hr when LCMS showed the reaction was complete. The reaction mixture was neutralized with 1N HCl and diluted with EtOAc (20 mL), washed with H2O (2×10 mL), concentrated and lyophilized to give rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid (19 mg, 42%). MS (ES⁺) m/z [(M+H)⁺]: 623.

### Example 7

### Preparation of rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

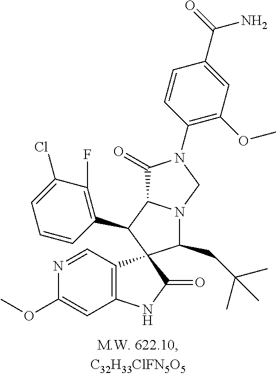

A mixture of rac-4-(3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid (14 mg, 0.022 mmol, Example 6) and N,N′-carbonyldiimidazole (Aldrich, 17 mg, 0.109 mmol) in THF (3 mL) was stirred at rt overnight. Ammonium hydroxide (108 mg, 0.12 mL, 0.912 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (2% to 12% EtOH in EtOAc) to give rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (9.7 mg, 68%) as a white solid, MS (ES⁺) m/z [(M+H)⁺]: 622

### Example 8

### Preparation of rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-2-methoxybenzamide

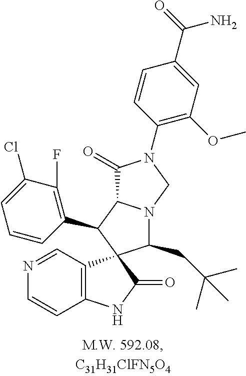 [SMILES: COc1cc(C(N)=O)ccc1N1CN2[C@@H](CC(C)(C)C)[C@@]3(C(=O)Nc4ccncc43)[C@@H](c3cccc(Cl)c3F)[C@@H]2C1=O]

Step a: To a solution of rac-(2S,3R,4S,5R)—N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide (20.7 mg, 0.035 mmol) and acetic acid (0.3 mL) in dichloromethane (0.5 mL), was added formaldehyde (contains 10-15% methanol as inhibitor) (22 mg, 0.020 mL, 0.27 mmol). The reaction mixture was heated under argon at 40° C. overnight. It was then poured into 2 N NaOH (4.5 mL) and extracted with EtOAc (2×20 mL). The organic layer was washed with water (10 mL) and concentrated to give crude rac-4-((3′R,5S,7S,7aR)-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-2-methoxybenzamide (23.9 mg) which was used without further purification.

Step b: The above crude rac-4-((3′R,5S,7S,7aR)-7-(3-chloro-2-fluorophenyl)-F-(hydroxymethyl)-5-neopentyl-1,2′-dioxo-1′,2′,7,7a-tetrahydro spiro[pyrrolo[1,2-c]imidazole-6,3′-pyrrolo[3,2-c]pyridine]-2(1H,3H,5H)-yl)-2-methoxybenzamide (23.9 mg, 0.035 mmol) was dissolved in EtOH (5 mL) and treated with a solution of 2N NaOH (0.48 mL, 0.96 mmol). The reaction mixture was heated to 50° C. and stirred for 2 hr when LCMS showed the reaction was complete. The reaction mixture was neutralized with 1N HCl and diluted with EtOAc (20 mL), washed with H2O (2×10 mL), concentrated and lyophilized to give rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-2-methoxybenzamide (5.3 mg, 25%).

MS (ES⁺) m/z [(M+H)⁺]: 592.

### Example 9

### Preparation of rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid

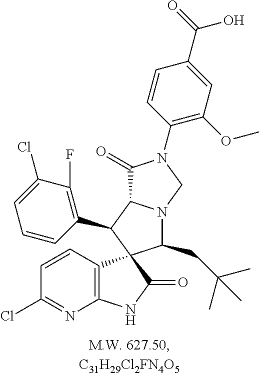 [SMILES: COc1cc(C(=O)O)ccc1N1CN2[C@@H](CC(C)(C)C)[C@@]3(C(=O)Nc4nc(Cl)ccc43)[C@@H](c3cccc(Cl)c3F)[C@@H]2C1=O]

Step a: To a solution of rac-methyl 442S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate (45 mg, 0.071 mmol) and acetic acid (0.52 mL) in dichloromethane (0.5 mL), was added formaldehyde (contains 10-15% methanol as inhibitor) (32 mg, 0.030 mL, 0.40 mmol). The reaction mixture was heated under argon at 40° C. overnight. It was then poured into 2 N NaOH (4.3 mL) and extracted with EtOAc (2×20 mL). The organic layer was washed with water (10 mL) and concentrated to give crude rac-methyl 4-((3′R,5S,7S,7aR)-6′-chloro-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoate (48 mg) which was used without further purification.

Step b: The above crude rac-methyl 443′R,5S,7S,7aR)-6′-chloro-7-(3-chloro-2-fluorophenyl)-1′-(hydroxymethyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoate (48 mg, 0.071 mmol) was dissolved in EtOH (5 mL) and treated with a solution of 2N NaOH (0.96 mL, 1.92 mmol). The reaction mixture was heated to 50° C. and stirred for 2 hr when LCMS showed the reaction was complete. The reaction mixture was neutralized with 1N HCl and diluted with EtOAc (20 mL), washed with H2O (2×10 mL), concentrated and lyophilized to give rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid (38.7 mg, 86.3%). MS (ES⁺) m/z [(M+H)⁺]: 627.

### Example 10

### Preparation of rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

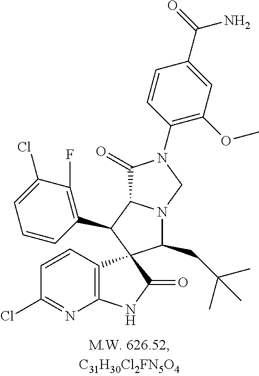 [SMILES: COc1cc(C(N)=O)ccc1N1CN2[C@@H](CC(C)(C)C)[C@@]3(C(=O)Nc4nc(Cl)ccc43)[C@@H](c3cccc(Cl)c3F)[C@@H]2C1=O]

A mixture of rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid (32 mg, 0.052 mmol, Example 9) and N,N′-carbonyldiimidazole (Aldrich, 37 mg, 0.232 mmol) in THF (7 mL) was stirred at rt overnight. Ammonium hydroxide (251 mg, 0.28 mL, 2.13 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (2% to 12% EtOH in EtOAc) to give rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzamide (18.8 mg, 57.8%) as a white solid, MS (ES⁺) m/z [(M+H)⁺]: 626

### Example 11

### Preparation of chiral 4-((3S,3′R,5S,7S,7aR)-6′-chloro-7-(3-chloro-2-fluorophenyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

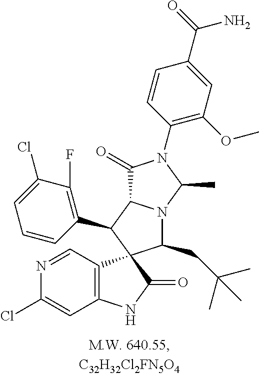 [SMILES: COc1cc(C(N)=O)ccc1N1C(=O)[C@H]2[C@H](c3cccc(Cl)c3F)[C@]3(C(=O)Nc4cc(Cl)ncc43)[C@H](CC(C)(C)C)N2[C@@H]1C]

Step a: To a solution of chiral (2S,3R,4S,5R)—N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide (21 mg, 0.034 μmol mmol) and acetic acid (0.5 mL) in dichloromethane (0.5 mL), was added acetaldehyde (27.5 mg, 0.035 mL, 0.624 mmol). The reaction mixture was heated under argon at 40° C. overnight. It was then poured into 2 N NaOH (4 mL) and water (5 mL) and extracted with EtOAc (10 mL). The organic layer was washed with water (8 mL) and concentrated to give crude chiral 4-((3S,3′R,5S,7S,7aR)-6′-chloro-7-(3-chloro-2-fluorophenyl)-1′-(1-hydroxyethyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (88 mg) which was used without further purification.

Step b: The above crude chiral 4-((3S,3′R,5S,7S,7aR)-6′-chloro-7-(3-chloro-2-fluorophenyl)-1′-(1-hydroxyethyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (87 mg, 0.034 mmol) was mixed with EtOH (3 mL) and treated with a solution of 2N NaOH (0.46 mL, 0.92 mmol). The reaction mixture was heated to 50° C. and stirred for 1 hr when LCMS showed the reaction was complete. The reaction mixture was poured into EtOAc (20 mL), washed with water (10 mL) and concentrated. The crude product was purified by flash chromatography (1% to 15% ethanol in dichloromethane) to give chiral 4-((3S,3′R,5S,7S,7aR)-6′-chloro-7-(3-chloro-2-fluorophenyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide as a white solid (12.5 mg, 56%). MS (ES⁺) m/z [(M+H)⁺]: 640

### Example 12

### Preparation of chiral 4-((3S,3′R,5S,7S,7aR)-6′-chloro-7-(3-chloro-2-fluorophenyl)-3-ethyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

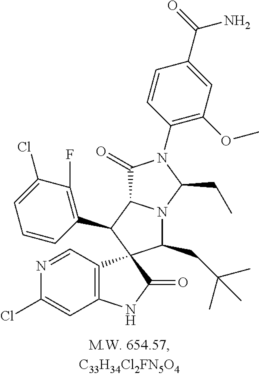 [SMILES: CC[C@@H]1N(c2ccc(C(N)=O)cc2OC)C(=O)[C@H]2[C@H](c3cccc(Cl)c3F)[C@]3(C(=O)Nc4cc(Cl)ncc43)[C@H](CC(C)(C)C)N12]

Step a: To a solution of chiral (2S,3R,4S,5R)—N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide (20 mg, 0.033 μmol mmol) and acetic acid (0.5 mL) in dichloromethane (0.5 mL), was added propionaldehyde (40 mg, 0.67 mmol). The reaction mixture was heated under argon at 40° C. overnight. It was then poured into 2 N NaOH (4 mL) and water (5 mL) and extracted with EtOAc (10 mL). The organic layer was washed with water (8 mL) and concentrated to give crude 4-((3S,3′R,5S,7S,7aR)-6′-chloro-7-(3-chloro-2-fluorophenyl)-3-ethyl-1′-(1-hydroxypropyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (44.5 mg) which was used without further purification.

Step b: The above crude chiral 4-((3S,3′R,5S,7S,7aR)-6′-chloro-7-(3-chloro-2-fluorophenyl)-3-ethyl-1′-(1-hydroxypropyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (44.5 mg, 0.033 mmol) was mixed with EtOH (3 mL) and treated with a solution of 2N NaOH (0.46 mL, 0.92 mmol). The reaction mixture was heated to 50° C. and stirred for 1 hr when LCMS showed the reaction was complete. The reaction mixture was poured into EtOAc (20 mL), washed with water (10 mL) and concentrated. The crude product was purified by flash chromatography (1% to 15% ethanol in dichloromethane) to give chiral 4-((3S,3′R,5S,7S,7aR)-6′-chloro-7-(3-chloro-2-fluorophenyl)-3-ethyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide as a white solid ((11.2 mg, 51%). MS (ES⁺) m/z [(M+H)⁺]: 654

### Example 13

### Preparation of chiral 4-((3S,3′R,5S,7S,7aR)-6′-chloro-7-(3-chloro-2-fluorophenyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

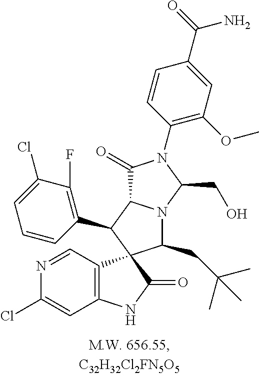 [SMILES: COc1cc(C(N)=O)ccc1N1C(=O)[C@H]2[C@H](c3cccc(Cl)c3F)[C@]3(C(=O)Nc4cc(Cl)ncc43)[C@H](CC(C)(C)C)N2[C@@H]1CO]

Step a: To a solution of chiral (2S,3R,4S,5R)—N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide (29.7 mg, 0.048 mmol) and acetic acid (1 mL) in dichloromethane (1 mL) in a microwave vial, was added (TERT-BUTYLDIMETHYLSILYLOXY)ACETALDEHYDE (183 mg, 0.2 mL, 0.945 mmol). The reaction mixture was heated under argon at 120° C. in the microwave for 30 min. It was then poured into 2 N NaOH (4 mL) and water (5 mL) and extracted with EtOAc (10 mL). The organic layer was washed with water (8 mL) and concentrated to give crude 4-((3S,3′R,5S,7S,7aR)-6′-chloro-7-(3-chloro-2-fluorophenyl)-1′-(1,2-dihydroxyethyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (162 mg) which was used without further purification.

Step b: The above crude chiral 4-((3S,3′R,5S,7S,7aR)-6′-chloro-7-(3-chloro-2-fluorophenyl)-1′-(1,2-dihydroxyethyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (162 mg) was mixed with EtOH (3 mL) and treated with a solution of 2N NaOH (0.65 mL, 1.3 mmol). The reaction mixture was heated to 50° C. and stirred for 1 hr when LCMS showed the reaction was complete. The reaction mixture was poured into EtOAc (20 mL), washed with water (10 mL) and concentrated. The crude product was purified by flash chromatography (1% to 15% ethanol in dichloromethane) to give chiral 4-((3S,3′R,5S,7S,7aR)-6′-chloro-7-(3-chloro-2-fluorophenyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide as a white solid (5.9 mg, 18.6%). MS (ES) m/z [(M+H)⁺]: 656

### Example 14

### Preparation of chiral 4-((3S,3′R,5S,7S,7aR)-6′-chloro-7-(3-chloro-2-fluorophenyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide

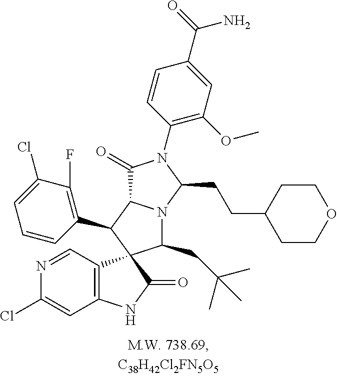 [SMILES: COc1cc(C(N)=O)ccc1N1C(=O)[C@H]2[C@H](c3cccc(Cl)c3F)[C@]3(C(=O)Nc4cc(Cl)ncc43)[C@H](CC(C)(C)C)N2[C@@H]1CCC1CCOCC1]

Step a: To a solution of chiral (2S,3R,4S,5R)—N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide ((21 mg, 0.034 mmol) and acetic acid (1 mL) in dichloromethane (1 mL) in a microwave vial, was added 3-(TETRAHYDRO-2H-PYRAN-4-YL)PROPANAL (24 mg, 0.169 mmol, Biofine Product List). The reaction mixture was heated under argon at 120° C. in the microwave for 1 h. It was then poured into 2 N NaOH (4 mL) and water (5 mL) and extracted with EtOAc (10 mL). The organic layer was washed with water (8 mL) and concentrated to give crude 4-((3S,3′R,5S,7S,7aR)-6′-chloro-7-(3-chloro-2-fluorophenyl)-1′-(1-hydroxy-3-(tetrahydro-2H-pyran-4-yl)propyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (33.1 mg) which was used without further purification.

Step b: The above crude chiral 443S,3′R,5S,7S,7aR)-6′-chloro-7-(3-chloro-2-fluorophenyl)-1′-(1-hydroxy-3-(tetrahydro-2H-pyran-4-yl)propyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide (33.1 mg) was mixed with EtOH (3 mL) and treated with a solution of 2N NaOH (0.47 mL, 0.94 mmol). The reaction mixture was heated to 50° C. and stirred for 1 hr when LCMS showed the reaction was complete. The reaction mixture was poured into EtOAc (20 mL), washed with water (10 mL) and concentrated. The crude product was purified by flash chromatography (1% to 15% ethanol in dichloromethane) to give chiral 4-((3S,3′R,5S,7S,7aR)-6′-chloro-7-(3-chloro-2-fluorophenyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide as a white solid (10.1 mg, 39%).

MS (ES) m/z [(M+H)⁺]: 738

### Example 15

### Preparation of rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid

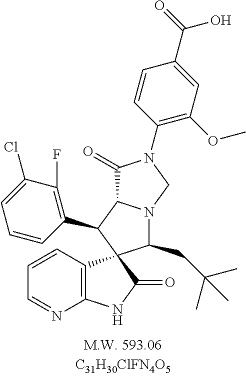 [SMILES: COc1cc(C(=O)O)ccc1N1CN2[C@@H](CC(C)(C)C)[C@@]3(C(=O)Nc4ncccc43)[C@@H](c3cccc(Cl)c3F)[C@@H]2C1=O]

The title compound was prepared in the similar manner as described in Example 4 starting from methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate and formaldehyde. MS (ES) m/z [(M+H)⁺]: 593.

### Example 16

### Preparation of rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid

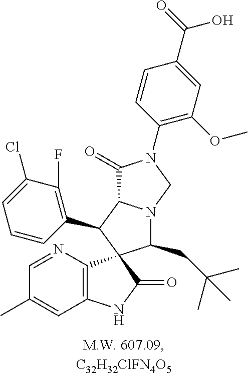

The title compound was prepared in the similar manner as described in Example 2 starting from rac-4-((2S,3 S,4S,5R)-4-(3-chloro-2-fluorophenyl)-6′-methyl-2-neopentyl-2′-o xo-1′,2′-dihydro spiro[pyrrolidine-3,3′-pyrrolo[3,2-b]pyridine]-5-ylcarboxamido)-3-methoxybenzoic acid and formaldehyde. MS (ES⁺) m/z [(M+H)⁺]: 607

### Example 17

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53. Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing:90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking. Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at 665 and 615 nm (Victor 5, Perkin ElmerWallac). If not specified, the reagents were purchased from Sigma Chemical Co.

Activity data for some of the Example compounds expressed as IC₅₀: bsa: 0.02% are as follows:

| Example Number | IC₅₀: bsa: 0.02% |
|---|---|
| 1 | 0.0068 |
| 2 | 0.1015 |
| 3a | 0.0899 |
| 3b | 1.1603 |
| 4 | 0.4845 |
| 5 | 0.7971 |
| 6 | 0.0230 |
| 7 | 0.0150 |
| 8 | 0.1033 |
| 9 | 0.0150 |
| 10 | 0.0193 |
| 11 | 0.0075 |
| 12 | 0.0070 |
| 13 | 0.0096 |
| 14 | 0.0075 |
| 15 | 0.0960 |
| 16 | 0.0218 |

What is claimed:

1. A compound of the formula 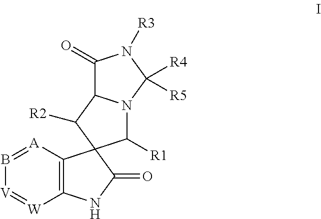
wherein 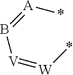
is selected from the group consisting of 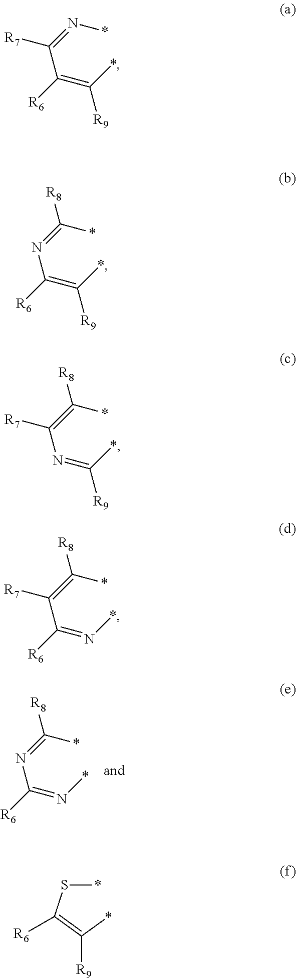
R₆ is selected from the group consisting of H, F, Cl, Br, I, CN, NO₂, ethynyl, cyclopropyl, lower alkyl, vinyl and alkoxy,
R₇ is selected from the group consisting of H, F, Cl, methyl;
R₈ is selected from the group consisting of H, F, Cl, methyl;
R₉ is selected from the group consisting of H, F, Cl, methyl;
wherein in the case of (f) A is a bond;
R₁ is independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ is selected from the group consisting of aryl, substituted aryl, heteroaryl and substituted heteroaryl;
R₃ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ— (CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group; and enantiomers thereof or a pharmaceutically acceptable salt or ester thereof.

2. A compound of the formula 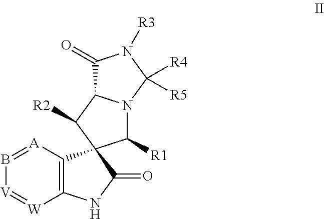
wherein 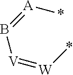
is selected from the group consisting of 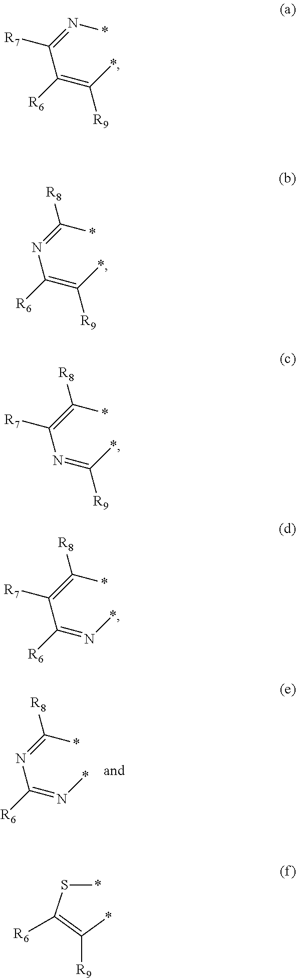
R₆ is selected from the group consisting of H, F, Cl, Br, I, CN, NO₂, ethynyl, cyclopropyl, lower alkyl, vinyl and alkoxy;
R₇ is selected from the group consisting of H, F, Cl, methyl;
R₈ is selected from the group consisting of H, F, Cl, methyl;
R₉ is selected from the group consisting of H, F, Cl, methyl;
wherein in the case of (f) A is a bond;
R₁ is independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ is selected from the group consisting of aryl, substituted aryl, heteroaryl and substituted heteroaryl;
R₃ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂′, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ— (CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ— (CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
and enantiomers thereof or a pharmaceutically acceptable salt or ester thereof.

3. The compound of claim 2 wherein R₆ is F, Cl or Br.

4. The compound of claim 3 wherein R₇, R₈, R₉ are all hydrogen.

5. The compound of claim 4 wherein R₂ is selected from the group consisting of aryl, aryl substitued with Cl or F or Br and heteroaryl optionally substituted with H, F, Cl or Br.

6. The compound of claim 5 wherein R₁ is a substituted lower alkyl of the formula 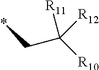
where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2 and
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle.

7. The compound of claim 6 wherein R₃ is (CH₂)ₙ—R′, n is 0 or 1 and R′ is aryl, substituted aryl, hetereoaryl, substituted heteroaryl, hetereocycle or substituted heterocycle.

8. The compound of claim 2 wherein
R₆ is selected from F, Cl or Br;
R₇, R₈, R₉ are hydrogen;
R₂ is selected from the group consisting of aryl, aryl substitued with Cl or F or Br, and heteroaryl optionally substituted with H, F or Cl or Br;
R₁ is a substituted lower alkyl of the formula 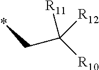
where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2;
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
R₃ is (CH₂)ₙ—R′;
n is 0 or 1;
R′ is selected from aryl, substituted aryl, hetereoaryl, substituted heteroaryl, hetereocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
and the enantiomers thereof or a pharmaceutically acceptable salt, or ester thereof.

9. A compound of the formula 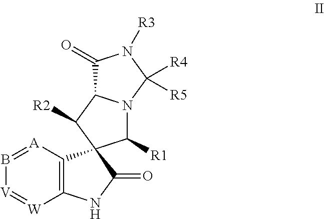
wherein 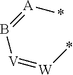
is selected from the group consisting of 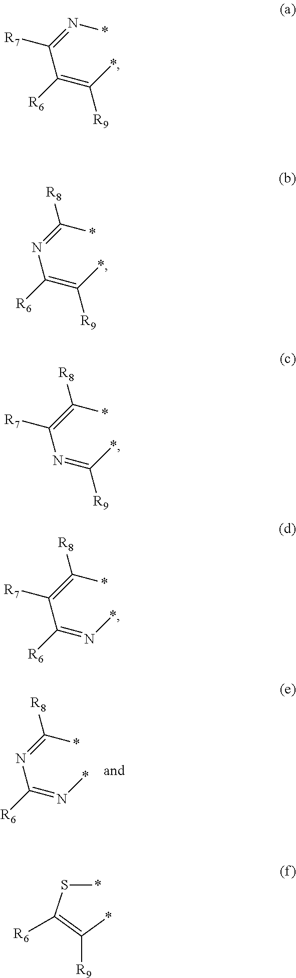
R₆ is selected from F, Cl or Br;
R₇, R₈, R₉ are hydrogen;
R₂ is selected from the group consisting of 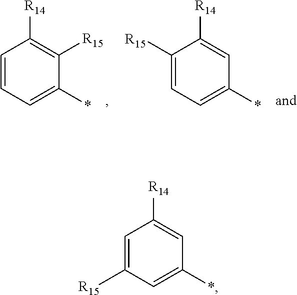
wherein
R₁₄ is F, Cl or Br;
R₁₅ is H or F;
R₁ is a substituted lower alkyl of the formula 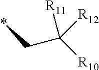
where R₁₀ and R₁₁ are both methyl, or alternatively, R₁₀ and R₁₁ together with the carbon to which they are attached form a ring selected from cyclopropyl, cyclobutyl, cyclopentyl or acyclohexyl;
R₁₂ is (CH₂)_{q}—R₁₃, where q is 0, 1 or 2;
R₁₃ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
R₃ is (CH₂)ₙ—R′;
n is 0 or 1;
R′ is selected from aryl, substituted aryl, hetereoaryl, substituted heteroaryl, hetereocycle or substituted heterocycle;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group;
or a pharmaceutically acceptable salt thereof.

10. A compound of claim 1 selected from the group consisting of
chiral 4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′S,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid,
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzoic acid,
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-2-methoxybenzamide and
rac-4-((3′R,5S,7S,7aR)-6′-chloro-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid.

11. A compound of claim 1 selected from the group consisting of
rac-4-((3′R,5S,7S,7aR)-7-(3-chloro-2-fluorophenyl)-6′-methoxy-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-methyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-ethyl-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide,
chiral 4-((3S,3′R,5S,7S,7aR)-6′-chloro-7-(3-chloro-2-fluorophenyl)-3-(hydroxymethyl)-5-neopentyl-1,2′-dioxo-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide;
chiral 4-((3S,3′R,5S,7S,7aR)-6′-chloro-7-(3-chloro-2-fluorophenyl)-5-neopentyl-1,2′-dioxo-3-(2-(tetrahydro-2H-pyran-4-yl)ethyl)-1′,2′,7,7a-tetrahydrospiro[pyrrolo[1,2-c]imidazole-6,3′-pyrrolo[3,2-c]pyridine]-2(1H,3H,5H)-yl)-3-methoxybenzamide;
rac-4-((3′R,5S,7S,7aR)-7-(3-chloro-2-fluorophenyl)-5-neopentyl-1,2′-dioxo-1′,2′,7,7a-tetrahydrospiro[pyrrolo[1,2-c]imidazole-6,3′-pyrrolo[2,3-b]pyridine]-2(1H,3H,5H)-yl)-3-methoxybenzoic acid and
rac-4-((3′S,5S,7S,7aR)-7-(3-chloro-2-fluorophenyl)-6′-methyl-5-neopentyl-1,2′-dioxo-1′,2′,7,7a-tetrahydrospiro[pyrrolo[1,2-c]imidazole-6,3′-pyrrolo[3,2-b]pyridine]-2(1H,3H,5H)-yl)-3-methoxybenzoic acid.

12. A pharmaceutical composition comprising a compound of the formula 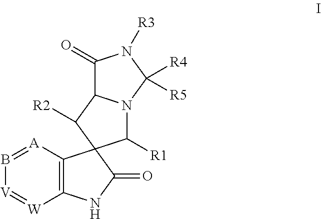
wherein 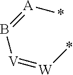
is selected from the group consisting of 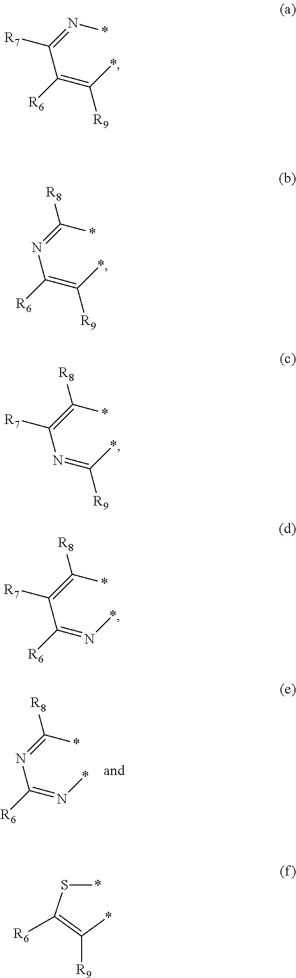
R₆ is selected from the group consisting of H, F, Cl, Br, I, CN, NO₂, ethynyl, cyclopropyl, lower alkyl, vinyl and alkoxy,
R₇ is selected from the group consisting of H, F, Cl, methyl;
R₈ is selected from the group consisting of H, F, Cl, methyl;
R₉ is selected from the group consisting of H, F, Cl, methyl;
wherein in the case of (f) A is a bond;
R₁ is independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₂ is selected from the group consisting of aryl, substituted aryl, heteroaryl and substituted heteroaryl;
R₃ is selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ (CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6;
one of R₄ and R₅ is H and the other is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl or R₄ and R₅ can be combined to form an oxo or thio group; and enantiomers thereof or a pharmaceutically acceptable salt or ester thereof
together with a pharmaceutically acceptable carrier or excipient.
